(19) United States
(12) Patent Application Publication
(10) Pub. No.: US 2005/0158723 A1
(43) Pub. Date: Jul. 21, 2005

(54) Irreversible colloidal chanis with recognition sites

(76) Inventors: Jean-Louis Viovy, Paris (FR); Jerome Bibette, Paris (FR); Cecile Goubault, Paris (FR); Marie Dutreix, L'Hay Les Roses (FR)

Correspondence Address: YOUNG & THOMPSON, 745 SOUTH 23RD STREET, 2ND FLOOR, ARLINGTON, VA, 22202, US

(21) Appl. No.: 10/504,776

(22) PCT Filed: Feb. 19, 2003
(86) PCT No.: PCT/FR03/00557

(30) Foreign Application Priority Data
Feb. 21, 2002 (FR) 02/02231

(51) Int. Cl. C12Q 1/68; G01N 33/551

(52) U.S. Cl. 435/6; 436/524

(57) ABSTRACT

A collection of colloidal particles in the form of one or several chains, in which the chains are generated in an irreversible manner and have at least one recognition site for a species, the site being different from sites implicated in the linear organisation of the particles. The invention further relates to a method for production of the collection, particularly for detection and/or dosage of at least one species in a fluid and a surface element functionalised by a collection of colloidal chains and a hybridisation network including such a surface element.

The invention relates mainly to magnetic colloidal particles organized in the form of permanent colloidal chains. It is also directed toward the use of these chains for detecting and/or analyzing specific species present in a fluid.

Methods for sorting and/or analyzing species contained in a liquid sample are already known. In general, they use either hybridization arrays arranged on surfaces like DNA or protein “chips”, or microbeads, or a combination of these two approaches. However, as emerges from the analysis below, these techniques have limitations.

In the “chips”, the biological ligands under consideration (DNA, oligonucleotides, proteins) are deposited or synthesized in situ in predetermined “spots” on a surface. Each spot has a typical surface area of 100 microns by 100 microns or less, which makes it possible to have a large number of recognition sites on a limited surface area, and therefore to carry out a large number of molecular analyses with a small amount of sample and in a limited period of time. Such systems, and also means for preparing them, are described, for example, in U.S. Pat. No. 5,744,305 (Affymetrix), Schena et al., Science, 270, 467-470, 1995. However, these chips have a sensitivity which remains insufficient for certain applications. What is more, their hybridization kinetics are significantly slowed down compared to a conventional hybridization in solution. Finally, they exhibit a lack of reproducibility. In this case, these disadvantages are based, to a large extent, on physicochemical aspects of the chips and of the hybridization mechanisms (see, for example, M. S. Shchepinov, S. C. Case-Green, E. M. Southern, Nucleic Acid Res. 25, 1155-61 (1997)).

The second method considered consists in binding the analytes to be tested, using a network of microspheres arranged on a surface. According to this technique, microspheres bearing various functionalities accessible at their surface can advantageously be prepared. However, the active surface area of a sphere remains, of course, of the same order of magnitude as that which it occupies on the surface of the test device. Consequently, this method does not therefore make it possible to obtain a significant gain in terms of sensitivity.

Magentic microbeads have also been proposed for analyzing oligonucleotides in a microfluid channel. These microbeads are introduced into a channel and retained at a site of said channel by a localized magnetic field: a zone essentially made up of a compact stack of magnetic beads is thus formed. The liquid containing the species to be analyzed is then made to circulate through this stack. The hybridization of said species is detected by fluorescence. Due to the circulation of the species, the kinetics are clearly more rapid than with the conventional DNA “chips”. Moreover, the system is recyclable, since, by eliminating the magnetic field, the beads become mobile again. However, the concentrations of analytes used to demonstrate the principle of the method are much greater than those really used in chips, which suggests that the sensitivity is low. This may in particular be explained by the compact assembly of beads. The beads closest to the detector form a screen for the transmission of the light to the others, and it is therefore only the beads closest to the surface which effectively participate in the detection.

Finally, microspheres are also used for identifying or analyzing species, and in particular biological species, in arrangements different from networks arranged on a surface or in a microfluid channel. They are in particular the techniques known as “magnetic sorting”, which can be used analytically or preparatively. In a very conventional method, a liquid containing the species to be analyzed (cells, DNA, proteins) is brought into contact with magnetic particles. The current version of these magnetic systems consists in introducing into the initial solution magnetic beads bearing functions specific for the cells to be isolated. After binding, the beads, and the species which are attached thereto, are pelleted using a magnet, whereas the supernatant is removed. This method is currently proposed essentially for the binary sorting of concentrated objects. It then requires an on-line analysis to produce the information. This magnetic method, which is simple to implement, has however two important limitations: incomplete selectivity and a purely binary nature, which both make it necessary to repeat the procedures when pure species or species corresponding to several criteria are sought. The lack of selectivity is caused first of all by the draining of the supernatant during the “sedimentation” of the particles, the moving beads displace with them part of the fluid and therefore the surrounding biological objects. The problems of nonspecific adhesion must then be taken into account, the force of magnetic pressure contributing to strongly anchoring to the beads any object trapped in the pellet.

For its part, the present invention aims to propose a novel tool for diagnosing and/or for preparing, identifying, analyzing or assaying species in a liquid sample, which gives satisfactory results both in terms of sensitivity, kinetics and reproducibility.

More precisely, a first subject of the present invention is an assembly of colloidal particles in the form of one or more chains, characterized in that said chains are organized irreversibly and have at least one recognition site for a species, said site being different from the ligands involved in the linear organization of said particles.

For the purpose of the invention, the term “chain of colloidal particles” or, without distinction, “colloidal thread” or “colloidal chain” is intended to mean an essentially linear assembly of colloidal particles. Various geometric organizations of such assemblies can be used in the context of the invention. In particular, it is possible to use a “pearl necklace” assembly in which the width of the chain is essentially that of a colloidal particle, or a “column” assembly, in which each section of the chain comprises several particles.

The colloidal chains according to the invention have an aspect ratio (ratio of the length to the largest dimension of a cross section) significantly greater than 1, typically greater than 3, and preferably greater than 5. For many applications, much higher aspect ratios, of 10 or more, or even greater than 100, can however prove to be advantageous.

The colloidal chains according to the invention can be relatively rigid (adopting essentially the form of a rod), semi-rigid (capable of having a radius of curvature comparable to their length), or flexible (capable of having a radius of curvature much smaller than their length). In the case of flexible chains, the length in the description above extends along the curvilinear abscissa of said chain. According to a preferred variant, they are semi-flexible or flexible.

In general, the cross section of the colloidal chains according to the invention is essentially circular. However, it may also have any other shape, provided that the largest dimension of this cross section remains smaller than the longest length of the colloidal chain, by a factor of at least 3.

For certain applications, it is possible to use a single colloidal chain of a given type, by analogy to that which is already used for individual molecules of DNA, in “single molecule” techniques. However, it is generally preferable to use a set of colloidal chains.

For the purpose of the invention, the term “colloidal particle” is intended to mean a compact three-dimensional object consisting of a multitude of atoms or of molecules, and capable of being maintained in suspension in a fluid. The dimensions of a colloidal particle are typically between a few tens of nanometers and a few microns, more rarely a few tens of microns. By way of example, latex spheres, microgels or magnetic beads of micron or submicron size, nanocrystals or microcrystals constitute colloidal particles according to the invention. Preferably, such particles are maintained in suspension by Brownian movement. However, particles which produce sedimentation can also be considered as colloidal for the purpose of the invention, provided that it is possible to resuspend them at the time of their use, for example by agitation or sonication.

The colloidal particles used to constitute the chains of colloidal particles according to the invention are preferably essentially spherical in shape.

These colloidal particles can be organic, mineral or organomineral. According to a preferred variant, they are completely or partly organic in nature, and preferably organomineral in nature, i.e. they have both organic constituents and mineral constituents.

Many types of organomineral particles are commercially available or known to those skilled in the art. They advantageously make it possible to combine properties derived from the organic portion and properties derived from the mineral portion, and therefore to construct colloidal chains according to the invention which have very diverse properties.

As regards the chemical nature of the mineral portion (or of the entire particle if it is essentially mineral), it can also be very varied, and can comprise in particular metal grains such as microparticles or nanoparticles of gold, of silver or of titanium, oxides of semiconducting metals, metal oxides, carbon particles, “quantum dots” with specific fluorescence or light absorption properties, and/or dielectric or conductive materials. Magnetic materials, such as superparamagnetic, ferrimagnetic, ferromagnetic or antiferromagnetic materials, or else conducting or semi-conducting materials, are most particularly suitable for the invention.

By way of oxides of semi-conductors, particles essentially consisting of silica or silicon oxide or comprising a silica shell are particularly advantageous.

This mineral portion can be either trapped at the heart of the colloidal particles making up the colloidal chain, or present at their surface. For example, to obtain conduction properties, it is possible, according to a first preferred variant, to have a metal layer over the surface of the particles. Conveniently, this layer can be obtained by means of a silver-type depositing process, such as the many known by those skilled in the art (see, for example, DNA-templated assembly and electrode attachment of a conducting silver wire, E. Braun, Y. Eichen, U. Sivan, G. Y. Ben-Yosph, Nature, 391, 775-778 (1998)). According to another preferred variant, a significant fraction of metal or semi-conducting grains may be included within the colloidal chain.

In the particular case of a magnetic material portion, it will be preferred for said portion to be located at the heart of the colloidal particles.

As regards the chemical nature of the organic portion, it can also be very diverse, and can comprise in particular, by way of example, plant, petroleum-based or synthetic oils, various polymers such as derivatives of acrylamide, of polystyrene or of polycarbonate, which may or may not be crosslinked, and, more generally, any of the materials used to constitute latices.

In particular, suitable for the invention are colloidal particles comprising a mineral core, coated with an organic layer of polymer type, such as, for example, polymers of polystyrene or polycarbonate type or derived from monomers of acrylic type, such as N-isopropylacrylamide, glycidyl acrylate or methacrylate, 2-hydroxyethyl methacrylate (HEMA) or ethylene dimethacrylate (EDMA). It may also be poly(methyl methacrylate). An organic shell is particularly advantageous in so far as it offers, via the presence of its surface organic functions, possibilities of grafting for recognition sites and/or secondary compounds and means for the linear organization of said particles. Any kinds of reactive functions, well known to those skilled in the art, can be used as surface reactive functions. By way of nonlimiting example, they may be carboxylic, amine, alcohol or thiol functions, polymerizable functions such as double or triple bonds, in particular allyl or acrylic functions, or else polyols, hydrazines or epoxides. They may also be ligands of biological type, such as biotin, streptavidin, avidin, digoxigenin or antidigoxigenin, and more generally antibodies or antigens commonly used as grafting sites in biology or else strong binding sites for transition metals, such as “histidine cages” for nickel.

The assemblies of colloidal particles claimed are organized linearly so as to form an irreversible chain or a set of irreversible chains of colloidal particles.

For the purpose of the present invention, the term “irreversible” is intended to characterize the inability of the linear chains of the colloidal particles to come apart spontaneously and/or after a brief period of time in the absence of an external field. In this case, excluded from the field of the invention are chains of particles for which the linear organization requires permanent maintenance of a magnetic or electric external field.

The irreversible nature of the assemblies of colloidal particles according to the invention is, on the other hand, taken to mean under given conditions of composition of the fluid in which they are suspended. Thus, such assemblies will be considered as irreversible even if it is possible to dissolve them by diluting them in a liquid having a composition or a pH significantly different from that of the liquid in which they were formed.

In the colloidal chains claimed, the cohesion between the particles can, in a preferred version, be maintained by covalent bonds between said particles, where appropriate resulting from bridging by means of molecules or macromolecules.

This covalent bond may involve specific interactions either directly between said particles or between the particles and molecules or macromolecules, via reactive functions present at the surface of these particles. The reactive functions may be amine, carboxylic acid, alcohol, aldehyde, thiol, epoxide or hydrazine functions and/or halogen atoms.

The constituting and the maintaining of a linear organization between the colloidal particles may also involve electrostatic, hydrophobic or Van der Waals interactions. To combine the colloidal particles with one another, it is also possible to involve specific interactions between said particles, that are different from those exerted with respect to the species to be analyzed or to be separated, either directly or by means of other molecules or macromolecules.

The assembly of particles claimed has at least one recognition site for a species and, preferably, several recognition sites of at least one given type.

The term “recognition site” is intended to mean a molecule, an ion, a surface element, or else a specific portion of a molecule or of an ion, capable of giving rise to an attractive interaction or to a chemical reaction with a particular species or a particular category of species.

Several distinct types of recognition sites can be carried by the same chain or on distinct chains when a set of chains is used. The number of types of sites may in particular be greater than 5 or than 10, or even, in certain applications, such as for example DNA or protein “chips”, from several hundred to several tens of thousands.

The recognition sites characterizing the colloidal chains according to the invention may be chosen, preferably, from nucleic acids (DNA, RNA, oligonucleotides), or synthetic analogs thereof (such as PNA, LNA, thiolated or methylated oligonucleotides), peptides, polypeptides, proteins, protein complexes, proteoglycans and polysaccharides. They may also be chosen from gene fragments, antibodies, antigens, enzymes or parts of enzymes, or biologically active parts of proteins, epitopes and haptens. However, as specified above, the type of recognition site considered for the purpose of detecting and/or assaying a species is different from the specific ligands involved, for their part, in the permanent organization of the colloidal particles in the form of a chain or chains. Thus, excluded from the field of the invention is a chain of colloidal particles in which the linear assembly is provided by the covalent coupling of a pair of ligands like, for example, the biotin/avidin pair, and which does not, moreover, have at least one recognition site other than the specific ligands of the pair under consideration, namely, in the example above, biotin or avidin.

The recognition sites present on the chains of particles claimed may also be chosen from chemical functions capable of specifically recognizing other chemical species, for example by bonding to them (for instance, by way of example, crown ethers capable of bonding transition metals, or vice versa), or by reacting with them (for instance, still by way of example, trypsins or alpha-chymotrypsins, capable of digesting proteins). They may also consist of ligands specific for metals, molecular footprints, catalytic sites, hydrophobic groups or, more generally, the functionalities used in chromatography to give columns a specific affinity for certain species. In particular, the recognition sites present on the chains of particles claimed may be chosen from compounds comprising aromatic or heterocyclic chemical functions, or sites capable of giving rise to hydrogen bonds.

For the purpose of the invention, the term “species” is intended to mean molecules or macromolecules, particles, atoms, ions, or objects of natural organic or artificial origin, such as nucleic acids, proteins, enzymes, antibodies, antigens, peptides, polypeptides, haptens, polysaccharides, proteoglycans, organelles, viruses, cells, sets of cells, microorganisms or colloids. They may also be nanoparticles or microparticles of natural or artificial origin, organic or organomineral molecules, drugs, medicinal products, or pollutants.

According to a preferred variant, a colloidal chain or a set of colloidal chains according to the invention has at least two distinct types of recognition sites.

In this case, an entirely unique advantage of the colloidal chains according to the invention is that, by virtue of their linear nature, they can have, along their backbone, various types of recognition sites. According to a preferred and very specific variant of the invention, these various types of sites are arranged in a predetermined (or sequenced) order along the colloidal chain(s) under consideration. Given the variety of accessible recognition sites, the ability to distinguish colloidal chains having the same recognition sites in a different order makes it possible to have a much richer combination than the conventional colloidal particles, which cannot involve sequences.

In addition, compared to spherical particles, the colloidal chains according to the invention have a better surface/volume ratio, at equal particle volume. In this case, by attaching the colloidal chains to a flat surface and/or within a channel, a much greater active surface is provided, compared to recognition sites deposited onto a surface, and this active surface can in particular extend over several tens of microns within said channel. This aspect of the invention is discussed in greater detail in the description hereinafter.

According to a preferred embodiment, the colloidal chains according to the invention also have one or more labels, which may be identical or different, that are especially useful for their detection.

Many labels of this type are known to those skilled in the art. A particularly advantageous family is that of the labels capable of interacting with electromagnetic radiation and, in particular, with visible, ultraviolet or infrared light, or else capable of emitting light under the action of a certain stimulus.

They may be labels capable of absorbing light within a certain wavelength range, or fluorescent or phosphorescent labels, such as molecules, molecular complexes or “quantum dots”. It may also be advantageous to use colloidal chains according to the invention which have molecules capable of electrochemical reactions (for instance, by way of example, hydroquinone and derivatives thereof), of electroluminescent effects or of chemiluminescent effects (electroactive or chemoactive compounds). By way of example, a certain number of horseradish peroxidase-based luminescent labels are well known to those skilled in the art and can be used in the context of the invention.

Advantageously, the colloidal chains according to the invention, as opposed to the conventional colloidal particles, lend themselves to the binding of one or more labels, which may be identical or different.

Of course, the embodiments described above can also apply to different recognition sites or to a combination of recognition sites and labels.

For certain applications, in particular for analyzing species or biological fluids, it may be advantageous for the colloidal chains according to the invention to also have on their surface molecules capable of preventing nonspecific adsorption phenomena. Such molecules are well known to those skilled in the art. They may in particular be hydrophilic polymers such as polyoxyethylene, polypropylene glycol, polysaccharides and, in particular, dextran or else polyacrylamide, or hydrophilic polymers of acrylamide, such as “Duramide”, poly-N-acryloylaminopropanol, poly-N-acryloylamino-ethanol, polyvinyl alcohol, polyvinylpyrrolidone, polydimethylacrylamide or copolymers of dimethylacrylamide and of allyl glycidyl ether. Such polymers can be grafted onto the surface of the colloidal chains according to the invention, either during the preparation of the initial particles or after the formation of said chains, using reactive functions integrated at the surface of said particles, or by direct adsorption.

In the context of the invention, the sets of colloidal chains characterized in that they are divided up into several colloidal chains, each chain having a given type of recognition site or of reactive function and, where appropriate, at least one given type of label, are particularly advantageous.

According to the applications, it is possible to use sets of colloidal chains which are substantially identical in length or, on the contrary, different in length.

According to a preferred variant, the colloidal chains in said set have a polydispersity in terms of length of less than 1.5, and preferably less than 1.2. The polydispersities are understood to be mass-averages.

According to another preferred variant, it is possible to use the length of the colloidal chains as a criterion for differentiation between two subfamilies and, therefore, to use, within a set of colloidal chains, several subfamilies of colloidal chains having different lengths, and essentially without any overlap of the size distribution between the various subfamilies. Preferably, in this variant, a correlation is established between the size of a colloidal chain and the type(s) of recognition sites that it has.

A second subject of the invention is a method that is useful for preparing an assembly of colloidal particles as claimed, characterized in that it comprises at least:
assembling colloidal particles in the form of one or more linear objects, and
bringing said objects into contact with at least one agent capable of irreversibly bridging them.

According to a preferred variant, this bringing into contact consists in migrating said agent in the vicinity of said objects.

In this case, the first step can be carried out by applying, transiently or permanently, to the colloidal particles an electric or magnetic field.

Thus, it is possible to confer a dipolar moment on colloidal particles in suspension, by means of an external field: the dipoles orient themselves in the direction of the field, attract along the axis of the field and repel in the perpendicular direction, thus constituting columns or “pearl necklaces”. According to one variant, it is possible to use a direct or alternating electric field, and particles which are in suspension in a medium and which exhibit an electric polarizability different from that of said medium.

According to a preferred variant, it is also possible to use magnetic particles which are aligned in a magnetic field. In this variant, superparamagnetic particles are particularly advantageous.

It is particularly convenient to perform the alignment of the colloidal particles, so as to constitute colloidal chains according to the invention, within a microfluid cell, and preferably within a channel or a chamber having at least two essentially parallel faces.

As regards the direction of the field, various geometries are possible. To obtain colloidal chains of uniform length, it is advantageous for the field serving to align the colloidal particles to be essentially uniform and perpendicular to said faces. To obtain very long colloidal chains, however, a configuration may be preferred in which the field is parallel to a direction in which the cavity within which the alignment is performed is large in size. In particular, it is, in this case, advantageous for the field to be parallel to the axis of the channel in which the alignment is performed or perpendicular both to this axis and to the smallest dimension of its cross section, if it is a parallelepipedal channel. When the field is parallel to the axis of the channel, it may be advantageous to adjust the density of colloidal particles such that a section of said channel contains, on average, only one object according to the invention or less.

As regards the step aimed at making the alignment of colloidal particles irreversible, various protocols and/or types of particles can be considered.

A first protocol consists in stabilizing superparamagnetic colloidal particles with a bridging agent of polymer type, and in particular a polyelectrolyte, for example of the polyacrylic acid type. In the absence of a magnetic field, or in the presence of a weak field, the particles exhibit, over a short range, a steric repulsion due to the polymer chains. For a magnetic field greater than a threshold field, the magnetic particles are pressed increasingly strongly against one another: some chains can cross this steric barrier, and can effect a bridging between the particles, which renders their association essentially irreversible or at least gives it a very long lifetime. An advantage of polyelectrolytes, besides their ability to interact strongly with particles of opposite charge, is that they can be brought into contact with the colloidal chains by means of an electric field.

A second protocol involves the assembly of the particles in columns by means of an external field within a cell having a semi-permeable wall, and the diffusion, across this wall, of a chemical agent capable of crosslinking the particles to one another.

It is also possible to use particles which bind to one another irreversibly under the simple action of an external field, without it being necessary to involve adjuvant molecules. Examples of this embodiment are given in Examples 8 and 9. This cohesion is interpreted as the result of hydrophobic interactions between the particles, which can become involved after crossing a barrier of repulsive potential under the action of the external field.

Finally, the alignment of the particles can be made irreversible using electrostatic interactions: it is possible, for example, to organize negatively charged (for example carboxylated) magnetic particles into filaments, in a magnetic field, and then to bring them into contact with polycations (for example using an electric field moving them in the opposite direction) the polycations, for instance, by way of example, polylysine or polyhistidine, attach to the particles and bridge them irreversibly, performing a “charge inversion” which converts the anionic reversible chain into a cationic irreversible chain. Examples of such embodiments are given in Example 12. The process can be repeated with a polyanion, such as polyacrylic or polyglutamic acid, which performs a second charge inversion. According to a particularly preferred variant, this second charge inversion can be obtained with a nucleic acid, which, at the same time, will play the role of recognition site and will therefore convert the simple irreversible colloidal chain into a colloidal chain according to the invention, i.e. having recognition sites. An example of such an embodiment is given in Example 7.

According to another embodiment, the method claimed comprises at least:
mixing colloidal particles and/or grafting them with at least one bridging agent or a bridging agent precursor,
assembling said colloidal particles in the form of one or more linear objects, and
initiating the bridging between said particles maintained in a linear organization.

In this case, it is possible to begin the process by constituting a colloidal chain, and then bridging the particles by means of electromagnetic radiation, for example in the context of a photochemical reaction. Use may, for example, be made of 5-azidonaphthalene-1-sulfonyl chloride, 4,4′-diazidostilbene-2,2′-disulfonic acid or, more generally, photoreactive crosslinking agents such as those described in the “Molecular Probes” catalog, chapter 5.3.

A photochemical reaction can also be used indirectly. For example, it is possible to form a mixture of magnetic colloidal particles having carboxylic functions, chains of polyamine in neutral form (for example, polylysine at a pH greater than 10.2), and orthonitrobenzyl, or more generally compounds comprising nitro or nitroso groups (see, for example, H. Morrisson, The chemistry of the Nitro and Nitroso groups, Feuer H. Ed., Interscience, New York, 1969, section I, chapter 4, or R. Bressauer, J. P. Paris, in Advances in Photochemistry, W. A. Noyes, G. S. Hammond, J. N. Pitts editors, Interscience, New York, 1963, p. 275, or else, R. W. Yip et al., J. Phys. Chem., 95, p. 6078 (1991) R53). The latter compounds, otherwise known as “proton cages”, are capable, under the action of ultraviolet radiation, of isomerizing and releasing a proton, creating an increase in pH sufficient to convert the neutral polyamine chain to polycation. The advantage of this method is that it makes it possible to avoid any aggregation between particles while the medium has not been subjected to light, and therefore to have the time to perform the mixing of the various constituents and to organize the colloidal particles in chains, before causing the light to act, which will create the attraction between the polymers and said particles, and the bridging of the latter in the desired linear configuration.

The bridging between colloidal particles can also be initiated by means of a change in temperature and/or a modification of pH.

Finally, if the initial particles spontaneously exhibit a short-range attractive potential and a long-range repulsive potential, it is possible to initiate the bridging by raising the external field (magnetic field for magnetic particles, electric field for dielectric particles).

According to a particular embodiment, the method claimed can be implemented in a microfluid cell comprising, besides a channel 1, in which the assembling of the colloidal particles or the functionalization thereof is performed, one or more secondary feed channels.

The organization of the colloidal particles into a chain can be carried out on particles having recognition sites and/or identical or different labels beforehand, as in Example 8 or, on the contrary, on nonfunctionalized particles, as in Examples 6 and 7. In the first case, the various particles having identical or different recognition sites and/or identical or different markers are organized so as to constitute said linear objects in a predetermined order. In this second case, the particles organized in linear chains are equipped with recognition sites and, optionally, with labels after constitution of said objects.

It is possible, of course, to combine the two variants above, i.e. to assemble, in a first step, colloidal particles having certain recognition sites and/or labels, and to add to these particles, in an order which may or may not be predetermined, other recognition sites and/or labels.

Be that as it may, the colloidal particles used for separating the colloidal chains have of course the specificities discussed above.

A third subject of the invention is a surface element bearing a linear assembly of colloidal particles according to the invention. In this case, it is particularly advantageous to have a surface exhibiting, at predefined sites, irreversibly assembled colloidal chains according to the invention.

Advantageously, the extension of these colloidal chains above the surface makes it possible to significantly increase, firstly, the specific surface for recognition and, secondly, the volume of supernatant solution brought into contact with the targets. In this case, the active surface area of said colloidal chains is greater than the surface area of the surface element bearing said chains and, preferably, by a factor of at least 4.

For example, with colloidal chains 200 nanometers in diameter, 1-micrometer equidistant, and 20 micrometers long, the specific surface area is 12 square micrometers per square micrometer of projected surface.

Preferably, the colloidal chains are attached to the surface by one of their ends. This attachment can be obtained by creating a covalent bond between the chains and the surface, by bridging with molecules or macromolecules and/or by electrostatic, hydrophobic or Van der Waals-type interactions. Specific interactions, different from those exerted between the recognition sites and the species, can also be envisioned.

By attaching the colloidal chains to the surface via one of their ends, a “colloidal brush” is formed. An example of such a surface is given in Example 5. This brush can be extended actively within the supernatant solution, for example by applying a magnetic field perpendicular to the surface of the “chip” if the chains comprise magnetic materials.

In the majority of applications, it is advantageous to bring the chains on said surface together in a multiplicity of distinct domains, preferably at predetermined positions on said surface. Preferably, the surface comprises at least two distinct domains comprising colloidal chains having different recognition sites.

Various methods can be used to constitute surfaces bearing a linear assembly according to the invention.

Typically, these methods should comprise the following steps:
grafting recognition sites onto the colloidal chains or onto at least some of the particles constituting said chains, and
attaching said colloidal chains to said surface.

Preferably, the step for attaching the colloidal chains to the surface is carried out in the presence of an external field capable of aligning said chains. For example, if the chains have an electric dipolar moment or electric polarizability, an electric field may be used to this effect. If they have a magnetic dipolar moment or magnetic polarizability, a magnetic field may be used.

According to a preferred variant, said field exhibits a multiplicity of local gradients, which direct the chains toward predetermined sites on the surface.

By means of these external fields, it is also possible to obtain surfaces on which the colloidal chains are rather oriented perpendicularly or are rather oriented parallel to the surface, depending on whether said field is rather perpendicular or rather parallel to the surface.

Optionally, these methods may also comprise a step for incorporating labels into said chains.

The order in which these various steps are carried out can vary according to the convenience of implementation. In particular, the grafting onto the surface can be prior to, simultaneously with, or subsequent to the placing of the recognition sites, subsequent to, simultaneously with, or prior to the placing of the labels. The placing of the labels, when this option is chosen, may, for its part, be subsequent to, simultaneous with or prior to that of the recognition sites.

Finally, it is to be noted that the assembling of the colloidal particles in chains may be prior to or simultaneous with the grafting thereof onto the surface. When this is possible, the second option is preferred, since it decreases the number of steps required for obtaining the final surface.

A subject of the invention is also a hybridization network comprising a surface element bearing colloidal chains according to the invention. This network may be low density, medium density or high density. These hybridization networks deposited onto a surface are generally referred to as “DNA chips”, “oligonucleotide chips” or “protein chips”.

In this type of application, the chains are grouped into a multiplicity of distinct domains on the surface element under consideration. Preferably, said domains occupy predetermined or pinpointable positions on said surface. Also preferably, at least two distinct domains comprise colloidal threads having different recognition sites. Preferably, there is a multiplicity of distinct domains each having a distinct type of recognition site. In certain cases, however, it may be desired to introduce a certain redundancy between the domains, for the purposes of controlling and/or measuring the reproducibility.

The “chips” formed from colloidal chains according to the invention exhibit many advantages compared to conventional chips: firstly, their specific surface area is increased, which increases the sensitivity, in particular in the case of competition with nonspecific ligands. The sample volume and therefore the number of species contained in the sample placed in the immediate proximity of the recognition sites is also considerably increased, which increases the kinetics and the sensitivity. Finally, in the case of magnetic colloidal chains, or more generally of colloidal chains sensitive to an external field, it is possible to agitate these colloidal chains with respect to the surrounding medium, for example by subjecting them to an oscillating external, magnetic or electric field, which makes it possible to accelerate the hybridization kinetics.

The use of colloidal chains according to the invention, of the type of those sensitive to an external field, also makes it possible to obtain more reproducible networks: in fact, the colloidal chains can be calibrated in length, and their physical self-organizational properties impose a uniform and predefined distribution over the entire surface of the domain.

Moreover, since the grafting of the recognition sites onto the filaments is carried out in batch, it can be controlled to a greater degree than in the case of conventional depositing or “spotting”, and can be the subject of a quality control before the depositing onto the surface.

A subject of the invention is also a microfluid cell or channel or a microcontainer containing an assembly of colloidal particles according to the invention. The term “microfluid cell” is intended to mean a device comprising a channel or a set of channels, one of the dimensions of which is between 100 nm and 1 mm, and which allows the transport of fluids.

According to a preferred variant, said chains are organized within the channel into a multiplicity of distinct domains. According to another preferred variant, which does not exclude the preceding variant, said chains are attached to one of the faces of the channel.

Finally, the colloidal chains according to the invention can also be used in an affinity electrophoresis, electrochromatography or chromatography device, in particular in order to act therein as a separation matrix. In this case, the analytes contained in a sample are introduced into said device. These analytes are transported therein within a channel, by means of a suitable field (pressure field for chromatography, electric field for electrophoresis or electrochromatography). The various analytes interact differently with the recognition sites present on the colloidal chains, and are therefore retained or slowed down to a greater or lesser degree. It is then possible to detect, by means well known to those skilled in the art (such as fluorescence, UV absorption, refractometry, electrochemistry, etc.), the various analytes after or during their passage between the colloidal chains. The differences in passage time provide information regarding the different affinities of the analytes with the colloidal chains and, where appropriate, regarding their nature. This method is particularly suitable for microfluid systems, by virtue of the most common dimensions of the colloidal chains according to the invention, which are in the micron range.

In another series of applications, the colloidal chains according to the invention can be used as “microreactors”. In this case, the recognition sites are catalytic sites, which make it possible to activate reactions with a very large surface/volume ratio. If the colloidal chains are used in bulk, they can, for example, be recovered easily by centrifugation or by magnetic sorting, and thus offer a good compromise between dissolved catalysts, which provide very good dispersion but are difficult to recover, and solid catalysts, which are easy to recycle by washing but relatively nondispersed. An example of an embodiment of a microreactor based on colloidal chains according to the invention is given in Example 13.

According to another particularly preferred variant, the claimed colloidal chains used as microreactors are attached to a surface, which makes it possible to exchange the reagents and to collect the reaction products as easily as with a solid catalyst, but with a much greater mobility and dispersion of the recognition sites.

The colloidal chains according to the invention are also advantageous for combinatorial chemistry applications. In the case in point, colloidal chains having enzymes as catalysts are particularly advantageous for combinatorial chemistry or diagnosis. Thus, it is possible, by way of nonlimiting example, to graft chymotrypsin or alpha-chymotrypsin onto magnetic colloidal particles according to the protocol described in Bilkova J., Chromatogr. A, 852, 141-149 (1999). These particles are then assembled by means of one of the methods described in Examples 1 to 8. It is also possible to first perform the assembling of the colloidal chains from microspheres of poly(HEMA-co-EDMA) type, to functionalize them with hydrazide, as described in Bilkova J., Chromatogr. A, 852, 141-149 (1999), and then to assemble them into colloidal chains according to one of the protocols described in Examples 1 to 8, and to repeat the protocol in step 2.9 of Bilkova J., Chromatogr. A, 852, 141-149 (1999) in order to attach the chymotrypsin in an oriented manner. Colloidal chains capable of digesting proteins are thus obtained.

A subject of the invention is also a molecular recognition microcontainer or device comprising colloidal chains according to the invention or a surface bearing such colloidal chains.

In fact, more generally, the colloidal chains according to the invention prove to be particularly advantageous for a large number of applications, such as the analysis, isolation and/or preparation of species.

Thus, a subject of the present invention is also a method for diagnosing and/or for analyzing, separating, purifying, assaying or identifying ex vivo at least one species, using at least one assembly of particles as claimed.

The species under consideration are those identified above.

These diagnostic and/or analytical methods can in particular be carried out according to the protocol comprising at least the steps consisting in:
a) using an assembly of colloidal particles within a channel or a container;
b) bringing said assembly into contact with at least one species to be detected, separated and/or assayed; and
c) using means for detecting the possible hybridization of the species under consideration with said assembly.

Optionally, in the preparative applications, it may also be advantageous to recover the products which have interacted with the colloidal chains or, conversely, those which have not interacted.

Also optionally, the process also comprises a washing step during which certain products contained in the sample and which have not hybridized with the chains are removed, or during which the products which have reacted with said chains are recovered.

For the purpose of the invention, the term “hybridization” is intended to mean any interaction in which a species binds specifically with a recognition site.

In the case of the claimed method, the chains of colloidal particles can be arranged in the form of distinct zones made up of several chains within a channel or a surface. In such a case, the zone containing said colloidal chains is generally crossed by a fluid containing at least one species to be analyzed.

The invention is particularly advantageous for this type of operation: in fact, during the washing, the colloidal chains can lie down and thus provide very little resistance to the flow, allowing easy and rapid washing. As soon as the flow is stopped, they can stand up again (this standing up can optionally be activated by a magnetic field, if the colloidal chains are magnetic) and again occupy a large volume. It is thus possible to combine the ease of washing obtained with open tubes, with the site density obtained with gels, but without the high resistance to the flow which the latter exhibit.

Among the means used in step c), fluorescence, phosphorescence, chemiluminescence, light absorption, surface plasmon resonance or radioactivity may preferably be used. It is also possible to use a method of detection employing a measurement of current, for example in one or more circuits included in the molecular recognition device, in the vicinity of the colloidal chain. The latter variant is particularly suitable for the case of current-conducting colloidal chains, or for colloidal chains using recognition sites capable of resulting in products that are detectable by an electrochemical reaction or a cyclic amperometry method. Use may also be made of one or more elements that are sensitive to the magnetic field or a change in magnetic field. The latter variant is particularly suitable for colloidal chains having magnetic properties.

As regards the detection, according to a first variant, the detection can be carried out in situ, within the channel or the container in which the hybridization, or more generally the interaction between certain species contained in a fluid and the recognition sites borne by the colloidal chains according to the invention, is carried out. According to a second variant, this detection can be carried out in another device, after the hybridization or interaction phase. In particular, use may be made of hybridization networks in accordance with the invention, in a manner comparable to conventional “DNA chips” or “protein chips”, by initially carrying out the hybridization in a hybridization chamber, and then carrying out the detection in a chip reader. When it is desired to simultaneously carry out a search for molecular recognition involving a multiplicity of types of colloidal chains, said chains can be organized in relatively compact (typically circular) zones or “spots”, or, on the other hand, in strips, within a channel or on a surface.

By way of nonlimiting example, the colloidal chains according to the invention, and the various components and devices using these colloidal chains, can be used for diagnosis; the search for and/or preparation of molecules or macromolecules, particles, atoms, ions, objects of natural organic or artificial origin, such as biologically active species, for instance nucleic acids, proteins, enzymes, antibodies, peptides, polypeptides, polysaccharides, proteoglycans, organelles, cancerous cells, rare cells, epithelial cells, endothelial cells, cells for prenatal diagnosis, GMOs, pathogenic cells, viruses, antibodies or microorganisms; the search for chemical active materials such as toxic products, drugs, or pollutants; the recognition of animal, plant or microorganism varieties; the detection of mutations; the search for allergies; genotyping; the search for genes involved in diseases; the search for and/or preparation of reaction products derived from combinatorial chemistry protocols.

The examples and figures below are given by way of nonlimiting illustration of the field of the invention.

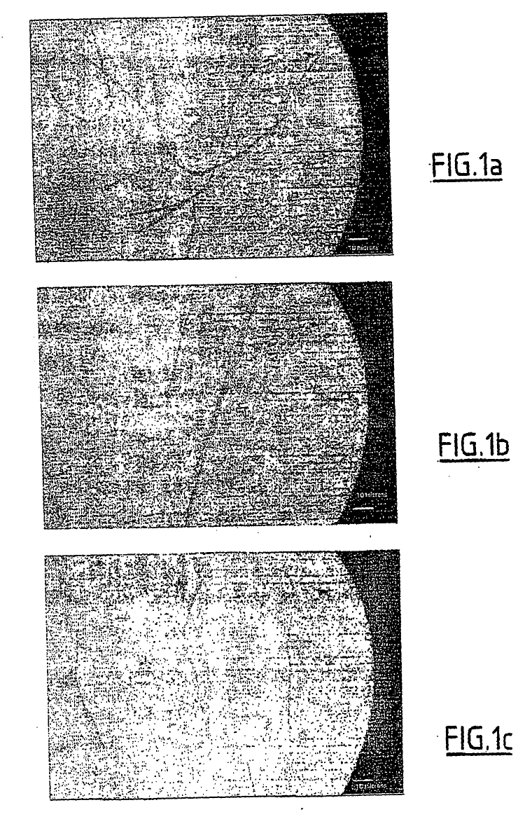

FIG. 1a: Example of flexible colloidal chains of the “pearl necklace” type, irreversibly bridged with polyacrylic acid, from magnetic particles of mean diameter 1.3 micrometers plus or minus 0.3 micrometers, prepared according to the protocol described in Example 1.

FIG. 1b: Example of rigid colloidal chains of the “column” type and of semi-flexible colloidal chains of the “pearl necklace” type, irreversibly bridged with polyacrylic acid, from magnetic particles of diameter 1.3 micrometers plus or minus 0.3 micrometers, prepared according to the protocol described in Example 1.

FIG. 1c: Example of monodispersed semi-flexible colloidal chains of the “pearl necklace” type, 70 micrometers long, organized irreversibly, according to the protocol described in Example 2.

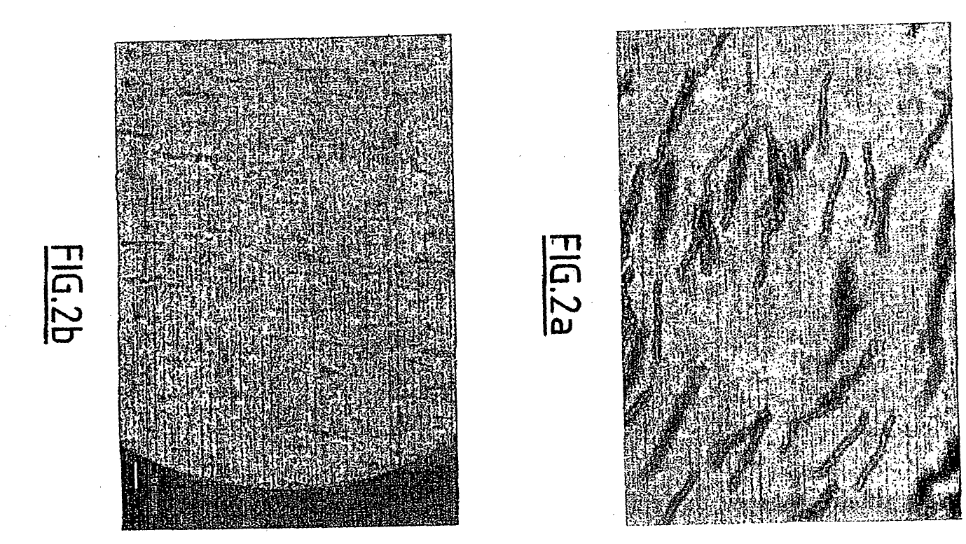

FIG. 2a: Example of colloidal chains bridged with polylysine, prepared according to Example 3.

FIG. 2b: Example of colloidal chains bridged with polylysine and attached to a surface by their end, prepared according to Example 4.

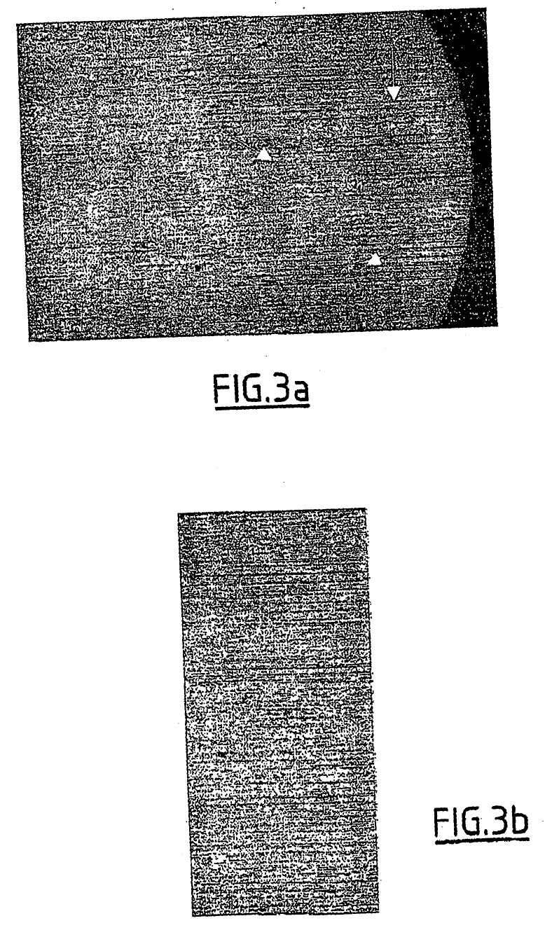

FIG. 3: Example of colloidal chains having DNA molecules: a/ chains having one DNA molecule per chain on average, prepared according to Example 6; b: colloidal chain having a uniform covering of “Phi X 174” DNA, prepared according to Example 7.

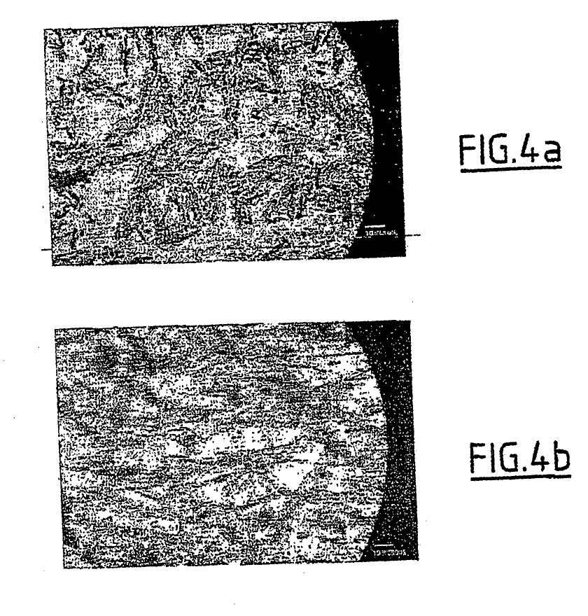

FIG. 4: Colloidal chains having antibodies: a/ colloidal chains having “anti-mouse” antibodies, prepared according to Example 8; b/ colloidal chains prepared from beads 1 micrometer in diameter, and having streptavidin, prepared according to Example 9a.

FIG. 5: Enzymatic protease activity of an assembly of colloidal particles according to the invention, prepared according to Example 13 and having trypsin recognition sites.

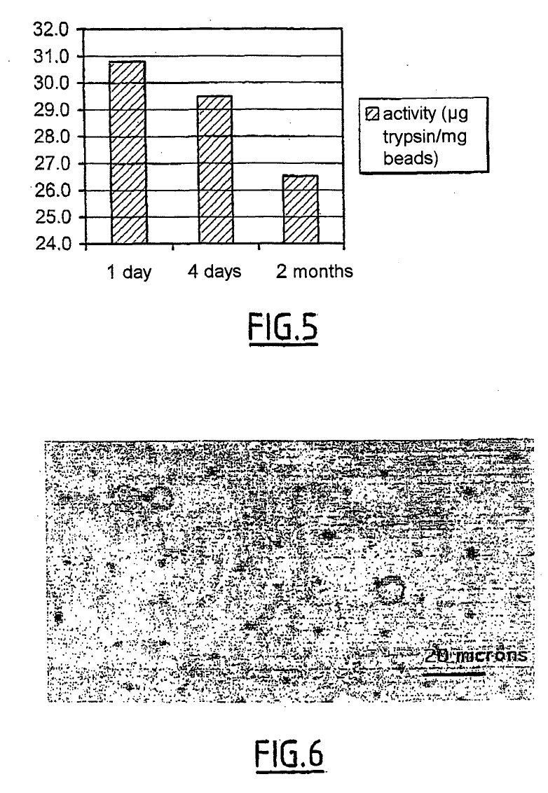

FIG. 6: Capture of erythrocyte cells having a biotin site, within a microchannel comprising a surface bearing assemblies of colloidal particles according to the invention having streptavidin recognition sites, prepared according to Example 14.

### EXAMPLE 1

Preparation of colloidal chains from particles 1.3 plus or minus 0.3 micrometers in diameter, using polyelectrolytes in the presence of a magnetic field, in a macroscopic container (test tube).

a/ Magnetic beads consisting of an inverse emulsion of octane-based ferrofluid (Rhone Poulenc), stabilized in water with sodium dodecyl sulfate, are prepared according to the protocol described in “Emulsions: theory and practice”, Becher, P., Rheinhold, New York, 1965). A particle size of 1.3 micrometers plus or minus 0.3 micrometers is selected by fractionated crystallization, according to the protocol described in Bibette, J. Colloid Interface Sci., 147, 474. (1991). According to one variant, commercially available magnetic particles, such as those distributed by the companies Bangs Laboratoires, Estapor, Merck, Eurolab, Prolabo, Uptima or Polysciences, can be used directly.

b/ The emulsion is washed several times (at least 5 times) with a solution of Nonyl Phenol Ethoxylate or NP10 (Sigma Aldrich) at 0.1%. The washing is carried out conveniently by pooling the magnetic drops at the bottom of the container with a magnet, replacing the supernatant with the washing solution, and vigorously agitating (sonication may optionally be used in the case of slight aggregation of the particles), after having withdrawn the magnet. The operation is repeated as many times as necessary. At the end of washing, an amount of NP10 solution to achieve a particle concentration of the order of 0.1% by volume is added.

c/ A solution of polyacrylic acid or PAA (Sigma Aldrich, Mw=250 000) is added in order to achieve a PAA concentration of 0.1%. The pH should be equal to 4. The mixture is left to incubate with gentle agitation.

d/ The test tube containing the sample is placed in a coil, and the magnetic field is gradually increased. Chains form, and the tube is left to incubate under the field for about 5 to 15 min. When a threshold field, of the order of 10 mT, has been exceeded, the chains remain irreversibly assembled after elimination of the magnetic field. Their average length and their diameter can be regulated by adjusting the amplitude of the magnetic field and the concentration of the magnetic particles. By way of example, the chains shown in FIG. 1a were obtained with a field of 50 mT, and a particle concentration of 0.1% by volume (observation between slide and cover, in the absence of magnetic field, under a Zeiss Axiovert 100 microscope using an immersion objective 100×, 1.3. Under these conditions, a coexistence between flexible chains of “pearl necklace” type (FIG. 1a), semi-flexible chains of “pearl necklace” type (FIG. 1b, on the left) and rigid chains of “column” type (FIG. 1b, on the right) is obtained. For lower particle concentrations, pearl necklaces are essentially obtained, and for higher concentrations, columns are essentially obtained. This method makes it possible to readily obtain large amounts of colloidal chains according to the invention. On the other hand, these chains are quite polydispersed.

### EXAMPLE 2

Production of Monodispersed Colloidal Chains of Calibrated Length

The procedure is carried out as in Example 1 up until step c.

For step d, instead of using a test tube, the solution is introduced into a channel having a uniform thickness of 100 micrometers, prepared by molding of polydimethylsiloxane according to Xia, Y. Xia, G. M. Whitesides, Angew. Chem. Int. Ed, 37, 550 (1998), and then a magnetic field of 50 mT is applied perpendicular to the thickness of the channel. After elimination of the magnetic field, semi-flexible chains of uniform length equal to the thickness of the channel (70 micrometers) are obtained (see FIG. 1c) (observation conditions identical to those of Example 1).

### EXAMPLE 3

Preparation of Colloidal Chains Bridged with Polylysine

a/ Magnetic particles are prepared according to a/ of Example 1.

b/ These particles are introduced into a channel prepared as in Example 2, by pressurization or capillarity, at a concentration which can vary, as needed, between 0.1% and 20%. A magnetic field of the order of 50 mT is applied perpendicular to the thickness of the channel. Colloidal chains are then obtained. These chains are of the “pearl necklace” type if the initial concentration of the suspension of magnetic beads is low (typically less than 5%), and of the “column” type if this initial concentration is high.

c/ Poly-L-lysine (0.1% w/v solution, Sigma Aldrich) is introduced into the channel by electrophoresis (for a volume fraction of beads of 2%, poly-L-lysine is introduced such that its concentration in the channel is 0.05 wt %). For this, two electrodes are provided in two reservoirs located at the ends of the channel. The polylysine is introduced, in the form of a solution, into one of the reservoirs, and the electrode located in this reservoir is brought to a positive potential with respect to that of the electrode located in the other reservoir, so as to maintain within the channel an electric field of a few V/cm. Irreversible colloidal chains such as those observed in FIG. 2a are obtained.

### EXAMPLE 4

Preparation of Colloidal Chains Attached by one of Their Ends to a Surface, Bridged with Polylysine (FIG. 2b)

a/ Magnetic particles are prepared or obtained as in step a of Example 1.

b/ The emulsion is washed several times (at least 5 times) with a solution of Nonyl Phenol Ethoxylate or NP10 (Sigma Aldrich) at 0.1%. The washing is carried out conveniently by pooling the magnetic drops at the bottom of the container with a magnet, replacing the supernatant with the washing solution, and vigorously agitating (sonication may optionally be used in the case of slight aggregation of the particles), after having withdrawn the magnet. The operation is repeated as many times as necessary. At the end of washing, an amount of NP10 solution to achieve a particle concentration of the order of 5% by volume is added.

c/ The continuous phase is replaced with a mixture consisting of 0.1 wt % NP10 and 0.89 wt % poly-L-lysine. For this, the magnetic beads are pooled at the bottom of the tube using a magnet, and the supernatant is removed and replaced with the desired mixture.

d/ The emulsion is introduced into a channel. A magnetic field of 50 mT is applied perpendicular to the thickness of the channel. After elimination of the magnetic field, a brush of calibrated chains attached to the lower wall of the channel is obtained.

### EXAMPLE 5

Preparation of Colloidal Chains Attached by One of Their Ends to a Surface, Coupled with Polydimethylacrylamide

a/ Magnetic particles are prepared or obtained as in step a of Example 1.

b/ A channel of uniform thickness is prepared by molding of polydimethylsiloxane according to Xia, Y. Xia, G. M. Whitesides, Angew. Chem. Int. Ed, 37, 550 (1998). This channel is filled with a solution of polydimethylacrylamide at 0.15% by mass, and is left to incubate for 40 min.

c/ The channel is rinsed with a solution of Triton X405 at 2.1 g/l, and is then filled with the suspension of magnetic particles prepared in a.

d/ The channel is placed at the center of a coil and, after equilibration of the pressures at the ends of the channel in order to avoid parasite flows, a magnetic field sufficient to create columns (of the order of one to a few tens of mT) is applied perpendicular to the thickness of the channel. The field is maintained for one hour.

e/ After elimination of the magnetic field, a “brush” of colloidal chains attached to the lower wall of the channel is obtained.

### EXAMPLE 6

Preparation of Colloidal Chains According to the Invention, Having One Molecule of “Lambda Phage” DNA Per Chain on Average.

a/ Colloidal chains are prepared according to the protocol described in Example 1.

b/ A solution of poly-L-lysine (0.1% w/v solution, Sigma Aldrich) is added so as to obtain in the mixture a poly-L-lysine concentration of 0.002 w %. The mixture is left to incubate with gentle agitation at ambient temperature for 40 min.

c/ When DNA is added to the suspension, it attaches at certain points of the surface of the colloidal chains, according to a mechanism probably comparable to that used on flat surfaces (see, for example, “The world of Microarrays, J. Boguslavs.ky, Drug Discovery and Development, S5-S32 (2001)). The concentration of DNA on the columns can be greatly varied, by adjusting the concentration of the DNA added to the solution, and its size. In FIG. 3a, approximately one large molecule of DNA (lambda phage, Amersham Pharmacia Biotech Inc) was attached per colloidal chain. The colloidal chains have a dark appearance and the DNAs labeled with a fluorescent marker (YOYO-1, Molecular Probes; one molecule of YOYO per ten base pairs) are light in appearance (measurement carried out by epifluorescence on a Zeiss Axiovert 100 microscope equipped with a mercury lamp for excitation and a 100× objective).

### EXAMPLE 7

Preparation of Colloidal Chains According to the Invention Having a Uniform Covering of DNA Molecules of “PhiX 174” type, Bound to the Colloidal Chains by Means of Polylysine

The procedure is carried out as in Example 6, but using DNA which is different in nature and a different DNA concentration for step c. A mixture of short DNAs of “PhiX 174” type is used (φX 174 RF DNA/Hae III Fragments; Gibco BRL). Ultimately, the concentration of DNA is 0.5 μg/ml and that of the poly-L-lysine is 0.002 wt %. The colloidal chains are then washed by pelleting them in a tube using a magnet, and replacing the supernatant with a solution identical to that used in b of Example 6. The observation conditions are the same as for Example 6, and result in colloidal chains uniformly covered with DNA (FIG. 3b).

### EXAMPLE 8

Production of Colloidal Chains According to the Invention, of the “Column” Type, Attached to a Surface and Having “Anti-Mouse” Recognition Functions

a/ A microfluid device comprising a channel of uniform thickness is prepared by molding of polydimethylsiloxane according to Xia, Y. Xia, G. M. Whitesides, Angew. Chem. Int. Ed. 37, 550 (1998).

b/ Anti-mouse Uptibeads (0.3 μm; Uptima), at the concentration of the original solution as sold by the manufacturer, are sonicated so as to break up the aggregates present in the initial sample, and introduced into the channel of the microfluid device prepared in a. This device is itself placed at the center of a coil so as to create within the channel an essentially uniform magnetic field oriented according to its thickness. For the observation, the device surrounded by its coil is placed on a Zeiss Axiovert 100 microscope and visualized using a 100×, 1.3. immersion objective and a Cohu CCD camera. A magnetic field of 50 mTesla is applied. When the magnetic field is eliminated, the particles remain grouped in the form of columns attached to the inner surface of the channel via one of their ends, and which can turn and orient themselves randomly around their point of attachment (figure a).

### EXAMPLE 9

Production, by Direct Bridging, of Colloidal Chains According to the Invention, of the “Column” Type, Having Streptavidin Functions

Streptavidin Uptibeads (0.88 μm), at the concentration of the original solution as sold by the manufacturer, are sonicated so as to break up the aggregates present in the initial sample, and placed in a microfluid cell as described in Example 8. When the magnetic field is eliminated, the particles remain grouped in the form of columns (FIG. 4b).

### EXAMPLE 10

Production of Colloidal Chains from Magnetic Particles Prefunctionalized with Galactose Oxidase in an Oriented Manner

a/ Preparation of the Magnetic Particles

Particles of HEMA-co-EDMA are prepared by polymerization in emulsion, and then activated with hydrazine, according to the protocol described in Horak et al., Biotechnol. Progr., 15 (1999).

b/ Preparation of Activated (Oxidized) Galactose Oxidase

A solution of galactose oxidase from Dactylium dendroides (350 IU) (Sigma Aldrich) is dissolved in 2.5 ml of 0.1 M acetate buffer, pH 5.5, containing 2 mM CuSO₄ and 1 mM of D-Fucose (Acros Organics, Geel, Belgium). 100 IU of catalase (Sigma Aldrich) are added. After incubation for 10 min at 37° C. and for 15 min at 4° C., 250 μl of NaIO₄ are added to the solution and agitated for 30 min at 4° C., so as to selectively activate the glycoside chains of the galactose oxidase. The reaction is stopped by adding 30 μl of ethylene glycol, and the agitation is continued for 10 min. The low molecular mass components are removed by filtration through a Sephadex G-25 column.

c/ Attachment of the Oxidized and Purified Galactose Oxidase to the Magnetic Particles

1.5 ml of solution of activated magnetic particles, prepared in a, are added to the purified galactose oxidase solution, such that the galactose oxidase is in excess with respect to the particle surface. Incubation is carried out for 24 h at 4° C. with agitation. The support is then washed with 0.1M acetate buffer, pH 4, containing 0.5M NaCl. The washing is repeated several times with a 0.1M phosphate buffer, pH 6, containing 2 mM CuSO₄, until the enzymatic activity of the eluent is no longer detectable (the oxidase activity is measured using a test based on the oxidation of D-galactose, as described in Avidad et al., J. Biol. Chem., 237, 2736 (1962)). The hydrazine groups which have not reacted are then blocked by incubation of the particles in a solution of 0.2M acetaldehyde in 0.1M acetate buffer, pH 5.5, for 24 h. Finally, the particles are equilibrated in a solution of 0.1M phosphate buffer, pH 6, containing 2 mM CuSO₄.

d/ Formation of the Colloidal Chains

Polyacrylic acid or PAA (Sigma Aldrich, Mw=250 000) is added to an aliquot of the solution of particles prepared in c, in order to achieve a concentration of PAA of 0.1%. The mixture is left to incubate with gentle agitation. The test tube containing the sample is placed in a coil and the magnetic field is gradually increased (N.B. If a coil which makes it possible to apply a sufficient magnetic field is available, the addition of PAA may be omitted, which makes it possible to leave the galactose oxidase functions more accessible and therefore to improve the catalytic yield of the final colloidal chains). Chains form and the tube is left to incubate under the field for about 15 min. After elimination of the magnetic field, irreversible colloidal chains having oxidase activity, demonstrated by spectrophotometric measurement using a test based on the oxidation of D-galactose, as described in Avidad et al., J. Biol. Chem., 237, 2736 (1962), are obtained.

### EXAMPLE 11

Functionalization of Colloidal Chains with Galactose Oxidase

a/ Preparation of the Magnetic Particles

A solution of colloidal magnetic particles is prepared, for example particles of HEMA-co-EDMA prepared according to Horak et al., Biotechnol. Progr., 15 (1999), or Ademtech particles. Polyacrylic acid or PAA (Sigma Aldrich, Mw=250 000) is then added so as to achieve a concentration of PAA of 0.1%. The mixture is left to incubate with gentle agitation. The test tube containing the sample is placed in a coil and the magnetic field is gradually increased. Chains form and the tube is left to incubate under the field for about 15 min. After elimination of the magnetic field, irreversible colloidal chains are obtained. (N.B. If a coil which makes it possible to apply a sufficient magnetic field is available, the addition of PAA can be omitted, which makes it possible to leave the galactose oxidase functions more accessible and therefore to improve the catalytic yield of the final colloidal chains). They are then activated with hydrazine according to the protocol described in Horak et al., Biotechnol. Progr., 15 (1999).

b/ Preparation of the Activated (Oxidized) Galactose Oxidase

A solution of galactose oxidase from Dactylium dendroides (350 IU) (Sigma Aldrich) is dissolved in 2.5 ml of 0.1 M acetate buffer, pH 5.5, containing 2 mM CuSO₄ and 1 mM of D-Fucose (Acros Organics, Geel, Belgium). 100 IU of catalase (Sigma Aldrich) are added. After incubation for 10 minutes at 37° C. and for 15 min at 4° C., 250 μl of NaIO₄ are added to the solution and agitated for 30 min at 4° C., so as to selectively activate the glycoside chains of the galactose oxidase. The reaction is stopped by adding 30 μl of ethylene glycol, and the agitation is continued for 10 min. The low molecular mass components are removed by filtration through a Sephadex G-25 column.

c/ Attachment of the Oxidized and Purified Galactose Oxidase to the Colloidal Chains

1.5 ml of solution of activated magnetic particles, prepared in a, are added to the purified galactose oxidase solution, such that the galactose oxidase is in excess with respect to the particle surface. Incubation is carried out for 24 h at 4° C. with agitation. The support is then washed with 0.1M acetate buffer, pH 4, containing 0.5M NaCl. The washing is repeated several times with a 0.1M phosphate buffer, pH 6, containing 2 mM CuSO₄, until the enzymatic activity of the eluent is no longer detectable. The hydrazine groups which have not reacted are then blocked by incubation of the particles in a solution of 0.2M acetaldehyde in 0.1M acetate buffer, pH 5.5, for 24 h. Finally, the colloidal chains are equilibrated in a solution of 0.1M phosphate buffer, pH 6, containing 2 mM CuSO₄. The oxidase activity is demonstrated by spectrophotometric measurement using a test based on the oxidation of D-galactose, as described in Avidad et al., J. Biol. Chem., 237, 2736 (1962).

### EXAMPLE 12

Preparation of Irreversible Columns of Magnetic Particles of the “Microreactor” Type, Having Trypsin Recognition Sites for the Digestion of Proteins

Chains of magnetic particles are prepared according to Example 1. The chains are rinsed and then resuspended in a phosphate buffer, pH 7.3, to which Nonyl Phenol has been added, in a proportion of 1 mg of magnetic particles in 400 microliters of buffer (solution A). The chains are sedimented carefully, keeping the magnet at least 2 cm from the tube. The trypsin is then immobilized according to a protocol derived from that described in the work by Greg T. Hermanson “Bioconjugate Techniques” 1996, Academic Press, London.

Furthermore, a solution B of 30 mg of ethylene carbodiimide (EDC) in 500 microliters of phosphate buffer, pH 7.3, is prepared.

A solution C of 5 mg of S-NHS(N-hydroxysuccinimide) in 400 microliters of phosphate buffer, pH 7.3, is also prepared.

Finally, a solution D is prepared: 7.5 mg of TPCK trypsin are dissolved in 50 microliters of phosphate buffer, pH 7, and 5 microliters of a solution of benzamidine at 16 micrograms per milliliter are added. Solution D is immediately added to solution A, without a magnetic field and while stirring gently with a Gilson pipette, the end of the tip of which has been cut off so as to decrease the shear. Solution B is then added, followed by solution C, still with gentle stirring. The solution is left to incubate for 3 hours and is then washed by means of 2 or 3 exchanges of buffer with a phosphate buffer, pH 7.3, containing Nonyl Phenol, identical to that of solution A. For the sedimentations, the procedure is carried out as described for the preparation of solution A.

The activity of the trypsin is measured using a calorimetric assay according to the protocol described in H. F. Gaertner and A. J. Puigserver, Enzyme Micro. Technol. 14, 150 (1992) and P. S. Gravet et al., Int. Biochem. 23, 1085 (1991).

A series of solutions of BAPNA (benzoylarginine p-nitroaniline HCl) at molar concentrations of between 0.1 and 1 are used as substrate, in a Tris buffer. The assemblies of particles having trypsin are introduced into the solution. After incubation for one hour, the amount of p-nitroaniline produced by the digestion reaction is measured by absorption of the solution at 410 nm, using a UV-Vis spectrophotometer (Shimadzu UV(160A)). The stability of the activity over time is given in FIG. 5.

In a variant, it is also possible to use, in the same protocol, magnetic beads based on silicon dioxide (SiO₂) (Kisker). Essentially mineral irreversible assemblies of colloidal particles according to the invention are then obtained. According to this variant, the attachment of a trypsin- or streptavidin-type recognition site is carried out by activation of the particles with glutaraldehyde.

### EXAMPLE 13

Preparation of Colloidal Chains Having Streptavidin Functions for Capturing Red Blood Cells Labeled with Biotin.

a/ A brush of colloidal chains having streptavidin functions is prepared in a channel, as described in Example 9.

b/ Human red blood cells are labeled with biotin according to the following protocol:
6 μl of blood are placed in 1 ml of 270 mOsmol PBS,
washing is carried out 3 times with a 0.1M carbonate/bicarbonate buffer, pH=8.5 (centrifugation at 3000 rpm for 1 min),
the pellet is taken and 500 μl of a solution of NHS-PEG 3400-biotin at 0.4 mg/ml are added,
incubation is carried out for 30 min,
centrifugation is carried out at 3000 rpm for 1 min in order to remove the supernatant, and 500 μl of PBS+0.5% BSA are added.

c/ The blood cells are then introduced into the channel and migrate through the brush of chains by means of an electric field, as described, for example, in Doyle et al., Science, 295, (5563), 2237, (2002). They attach to the columns of magnetic particles, as is demonstrated in FIG. 6.

1-44. (canceled)

45. An assembly of colloidal particles in the form of one or more chains, wherein said chains are organized irreversibly and have at least one recognition site for a species, said site being different from the ligands involved in the linear organization of said particles.

46. The assembly of colloidal particles as claimed in claim 45, wherein the colloidal particles are essentially spherical in shape.

47. The assembly as claimed in claim 45, wherein said chains are flexible or semi-flexible.

48. The assembly as claimed in claims 45, wherein said chain has an aspect ratio of greater than 1, and preferably greater than 3.

49. The assembly as claimed in claim 45, wherein said particles are totally or partly organic in nature, and preferably organomineral in nature.

50. The assembly as claimed in claims 45, wherein said particles are essentially mineral in nature.

51. The assembly as claimed in claim 50, wherein said particles essentially consist of silica or comprise a silica shell.

52. The assembly as claimed in claim 45, wherein said particles are based on a ferromagnetic, ferrimagnetic, antiferromagnetic, superparamagnetic, conducting or semi-conducting material.

53. The assembly as claimed in claim 45, characterized in that the colloidal particles comprise a mineral core coated with a polymeric organic layer.

54. The assembly as claimed in claim 45, wherein the cohesion between said particles is maintained by covalent bonds between said particles.

55. The assembly as claimed in claim 45, wherein the cohesion between the particles results from bridging by means of molecules or macromolecules.

56. The assembly as claimed in claim 54, wherein the cohesion between the particles involves specific interactions directly between said particles or with molecules or macromolecules, via reactive functions present at the surface of said particles.

57. The assembly as claimed in claim 56, wherein the reactive functions are amine, carboxylic acid, alcohol, aldehyde, thiol, epoxide or hydrazine functions and/or halogen atoms.

58. The assembly as claimed in claim 55, wherein the cohesion between said particles involves interactions of electrostatic, hydrophobic or Van der Waals type.

59. The assembly as claimed in claim 45, wherein the recognition site(s) is (are) chosen from:
nucleic acids or synthetic analogs thereof, peptides, polypeptides or proteins, protein complexes, proteoglycans, polysaccharides, gene fragments, antibodies, antigens, enzymes, epitopes, haptens, chemical functions capable of specifically recognizing other chemical species, ligands specific for metals, catalystic sites, molecular footprints, hydrophobic groups, enzymes or parts of enzymes.

60. The assembly as claimed in claim 45, wherein the recognition site(s) is (are) molecules, ions, surface elements, or else specific portions of a molecule or of an ion that are capable of giving rise to an attractive interaction or to a chemical reaction with a particular species or a particular category of species.

61. The assembly as claimed in claim 60, wherein the recognition site(s) is (are) chosen from compounds comprising aromatic or heterocyclic chemical functions or sites capable of giving rise to hydrogen bonds.

62. The assembly as claimed in claim 45, wherein said particles are organized in the form of a single chain or of a set of colloidal chains having at least two distinct types of recognition sites.

63. The assembly as claimed in claim 45, wherein the various types of recognition sites, or of functions, are organized in a predetermined order along the chain(s) under consideration.

64. The assembly as claimed in claim 45, wherein said particles or some of them have one or more labels which may be identical or different.

65. The assembly as claimed in claim 45, wherein it consists of several chains, each chain having a given type of recognition site or of reactive functions and, where appropriate, at least given type of label.

66. A method that is useful for preparing an assembly of colloidal particles as claimed in claim 45, comprising the steps consisting of at least:
assembling colloidal particles in the form of one or more linear objects, and
bringing said objects into contact with at least one agent capable of irreversibly bridging them.

67. The method as claimed in claim 66, wherein the bridging agent is chosen from polymers, and preferably polyelectrolytes.

68. The method as claimed in claim 66, wherein the linear assembly of said particles is obtained by transient or permanent action of a magnetic field or of an electric field.

69. The method as claimed in claim 66, wherein the organization of said particles is carried out in a microfluid cell or within a channel or a chamber having at least two essentially parallel faces.

70. A method that is useful for forming an assembly of colloidal particles as claimed in claim 45, comprising the steps consisting in at least:
mixing colloidal particles and/or grafting them with at least one bridging agent or a bridging agent precursor,
assembling said colloidal particles in the form of one or more linear objects, and
initiating the bridging between said particles maintained in a linear organization.

71. The method as claimed in claim 70, wherein the third step involves a modification of temperature, application of electromagnetic radiation of an electric field or of a magnetic field, a change in pH and/or a photochemical reaction.

72. A method for diagnosing and/or for analyzing, purifying, identifying, separating or assaying ex vivo at least one species, using at least one assembly of colloidal particles as claimed in claim 45.

73. The method as claimed in claim 72, wherein the species are chosen from proteins, nucleic acids, synthetic equivalents of nucleic acids, proteoglycans, haptens, enzymes, antibodies, antigens, synthetic macromolecules, pollutants, organelles, cells, viruses, microorganisms, nanoparticles or microparticles of natural or artificial origin, organic or organomineral molecules, drugs and medicinal products.

74. The method as claimed in claim 72, comprising the steps consisting in at least:
using, within a channel or a container, an assembly of colloidal particles in the form of irreversible chains;
bringing said assembly into contact with at least one species to be separated, detected and/or assayed, and
using means for detecting the possible hybridization of the species under consideration with said assembly.

75. The method as claimed in claim 74, further comprising a washing step during which the products which have not hybridized with said chains are removed, or during which the products which have reacted with said chains are recovered.

76. The method as claimed in claim 74, wherein the chains of colloidal particles are arranged in the form of several distinct zones, within a channel or on a surface.

77. The method as claimed in claim 76, wherein the zone containing said chains is crossed by the fluid containing at least one species to be analyzed.

78. The use of an assembly of colloidal particles as claimed in claim 45, in an electrochromatography, affinity electrophoresis or chromatography device.

79. The use of an assembly of colloidal particles as claimed in claim 45, as microreactors.

80. The use of an assembly of colloidal particles as claimed in claim 79, wherein the recognition site attached to said particles is a catalytic site.

81. The use of an assembly of colloidal particles as claimed in claim 45, in combinatorial chemistry.

82. A surface element bearing an assembly of colloidal particles as claimed in claim 45.

83. The surface element as claimed in claim 82, wherein the active surface area of said colloidal chains is greater than the surface area of the surface element bearing said chains.

84. The surface element as claimed in claim 82, characterized in that said chains are bound to said surface via one of their ends.

85. The surface element as claimed in claim 82, wherein the attachment of said chains to said surface is obtained by creating a covalent bond between the chains and the surface, bridging by means of molecules or macromolecules, and/or by electrostatic interactions of hydrophobic or Van der Waals type.

86. The surface element as claimed in claims 82, wherein said chains are assembled, on said surface, into at least two distinct domains comprising colloidal chains having different recognition sites.

87. A hybridization network comprising a surface element as claimed in claim 82.

88. A microfluid cell or channel comprising an assembly of colloidal particles as claimed in claim 45.
